(19) United States
(12) Patent Application Publication
(10) Pub. No.: US 2025/0269009 A1
(43) Pub. Date: Aug. 28, 2025

(54) VACCINE COMPOSITION FOR NOVEL CORONAVIRUS INFECTION

(71) Applicants: RONGSEN BIOTECHNOLOGY (BEIJING) CO., LTD, Beijing (CN); INSTITUTE OF BIOPHYSICS, CHINESE ACADEMY OF SCIENCES, Beijing (CN)

(72) Inventor: Baidong HOU, Beijing (CN)

(21) Appl. No.: 18/267,355

(22) PCT Filed: Dec. 15, 2021
(86) PCT No.: PCT/CN2021/138348

§ 371 Date: Jun. 14, 2023

(30) Foreign Application Priority Data
Dec. 15, 2020 (CN) 202011480978.4

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/5258; A61K 2039/575; C07K 2319/20; C12N 2770/20022; C12N 2770/20034; C12N 2795/18022; C12N 2795/18023

(57) ABSTRACT

The present application relates to a novel coronavirus pathogen-like antigen (PLA) vaccine, a preparation method therefor and an application thereof. The PLA vaccine consists of structurally-modified Escherichia coli virus-like particles (VLPs) and novel coronavirus antigens displayed thereon, and nucleic acid is encapsulated inside of the VLPs. The novel coronavirus PLA vaccine of the present invention formed by passing through modifications effectively prevents the aggregation or precipitation of particles, facilitating the production of the vaccine and ensuring the stability of vaccine efficacy; in addition, relative to conventional vaccines which require additionally adding an additional adjuvant, the PLA-SARS-CoV2 vaccine of the present invention is capable of inducing a significantly higher level of specific antibodies and neutralizing antibodies, having a significantly higher efficacy in challenge tests relative to conventional vaccines.

### SEQUENCE LISTING

The instant application contains a Sequence Listing which has been submitted electronically in ASCII format and is hereby incorporated by reference in its entirety. Said ASCII copy, created on Jan. 26, 2024, is named 51728-004001_Sequence_Listing_1_26_24_ST25 and is 19,982 bytes in size.

### TECHNICAL FIELD

The present invention belongs to the field of biological vaccines, in particular to a pathogen-like antigen vaccine composition for preventing and/or treating SARS-Cov2 infection.

### BACKGROUND OF THE INVENTION

Novel coronavirus disease (Corona Virus Disease 2019, COVID-19) is an acute respiratory infectious disease caused by novel coronavirus (SARS-Cov2) infection. SARS-Cov2 is highly infectious and the population is generally susceptible, so it spreads quickly. Meanwhile, due to the complexity of the virus infection mechanism and the risk of a long-term epidemic, it has increased the difficulty of epidemic prevention and control. Vaccines are the most effective and economical means of controlling infectious diseases. There are currently hundreds of COVID-19 vaccines under urgent development around the world. The vaccine construction strategies adopted are different, and they can be divided into five types according to technical characteristics, including: 1) inactivated virus vaccines; 2) viral vector vaccines, such as adenovirus vector vaccines; 3) mRNA vaccines; 4) DNA vaccines and 5) protein subunit vaccines, such as vaccines using SARS-CoV2 S1 protein or RBD protein mixed with adjuvants. These five types of vaccines have different mechanisms of action, but there are still various defects and uncertainties in terms of effectiveness and safety. Therefore, there is currently no guarantee that these vaccine strategies can produce high-quality vaccines that meet the needs of COVID-19 global epidemic prevention and control.

According to the requirements put forward by the FDA in the United States and other agencies, COVID-19 vaccines need to meet various performance requirements, including: 1) effectiveness: the protective power is required to be greater than 50%; 2) safety: avoiding risks such as ADE/ERD; 3) providing long-lasting immune protection. In addition, due to the huge number of people who need to be vaccinated and protected, vaccine development also requires: 1) low requirements for vaccine storage and transportation, such as 4° C. storage conditions; 2) economical, easy to produce on a large scale and at low cost. Finally, in order to cope with the immune escape caused by mutations in SARS-Cov2, an ideal vaccine strategy needs to be able to quickly adapt to the impact of virus mutations.

### SUMMARY OF THE INVENTION

To this end, in one aspect, the present invention relates to a soluble pathogen-like antigen complex comprising: (1) a virus-like particle (VLP), which is self-assembled by a first fusion protein comprising a viral capsid protein or a variant thereof at its N-terminus and SpyTag at its C-terminus, (2) a second fusion protein, comprising an antigen from S1 protein of SARS-CoV2 virus or a variant thereof and SpyCatcher, preferably the SpyCatcher is at the N-terminus of the second fusion protein; wherein, the virus-like particle also encapsulates nucleic acid inside it, and wherein the virus-like particle and the antigen from the S1 protein of SARS-CoV2 virus are covalently connected through SpyCatcher and SpyTag, so that the antigen from S1 protein of SARS-CoV2 virus or a variant thereof is displayed on the surface of the virus-like particle.

The soluble pathogen-like antigen complex of the present invention, wherein the nucleic acid encapsulated in the virus-like particle is the nucleic acid which is encapsulated by the virus-like particle when self-assembling and is from host bacteria used to express the virus-like particles, preferably the host bacteria is Escherichia coli, and preferably the nucleic acid is RNA.

The soluble pathogen-like antigen complex of the present invention, wherein the capsid protein is from Escherichia coli phage Q3, MS2 or AP205, preferably from Escherichia coli phage AP205.

The soluble pathogen-like antigen complex of the present invention, wherein the sequence of the capsid protein of the phage AP205 has at least 90%, 91%, 92%, 93%, 94%, 95% %, 96%, 97%, 98%, 99% or 100% identity with SEQ ID NO: 1.

The soluble pathogen-like antigen complex of the present invention, wherein the sequence of the capsid protein of the phage AP205 is SEQ ID NO: 1.

The soluble pathogen-like antigen complex of the present invention, wherein the antigen is a RBD sequence of the S1 protein of SARS-CoV2 virus.

The soluble pathogen-like antigen complex of the present invention, wherein the sequence of the antigen has at least 90%, 91%, 92%, 93%, 94%, 95%, 96%, 97% %, 98%, 99% or 100% identity with SEQ ID NO: 2.

The soluble pathogen-like antigen complex of the present invention, wherein the sequence of the antigen is SEQ ID NO: 2 or SEQ ID NO: 24.

The soluble pathogen-like antigen complex of the present invention, wherein the sequence of SpyTag has at least 90%, 91%, 92%, 93%, 94%, 95%, 96%, 97%, 98%, 99% or 100% identity with of SEQ ID NO: 3, and the sequence of SpyCatcher has at least 90%, 91%, 92%, 93%, 94%, 95%, 96%, 97%, 98%, 99% or 100% identity with SEQ ID NO: 4.

The soluble pathogen-like antigen complex of the present invention, wherein in the first fusion protein, the phage capsid protein or a variant thereof is linked with SpyTag via a first linker peptide, and in the second fusion protein, the antigen or a variant thereof is linked to SpyCatcher via a second linker peptide.

The soluble pathogen-like antigen complex of the present invention, wherein the sequence of the first linker peptide is SEQ ID NO: 5, and the sequence of the second linker peptide is SEQ ID NO: 6.

The soluble pathogen-like antigen complex of the present invention, wherein the second fusion protein is connected to the virus-like particle at a ratio of less than or equal to 1:1, preferably connected at a ratio of 1:6-1:12, and preferably connected at a ratio of 1:6, 1:7, 1:8, 1:9, 1:10, so as to ensure the solubility and immunogenicity of the pathogen-like antigen complex, the ratio is calculated as the ratio of SpyCatcher on the second fusion protein to SpyTag on the virus-like particle.

In another aspect, the present invention relates to a pathogen-like antigen vaccine composition, comprising the soluble pathogen-like antigen complex of the present invention and pharmaceutically acceptable carriers and/or excipients.

In a further aspect, the present invention relates to a method for preventing and/or treating diseases associated with SARS-CoV2 virus infection in a subject in need thereof, wherein a preventive and/or therapeutically effective amount of the soluble pathogen-like antigen complex or vaccine composition of the present invention is administered to the subject.

The PLA-SARS-CoV2 vaccine of the present invention has the following advantages:
(1) It can initiate the immune response by regulating the antigen presentation function of B cells with the advantages of strong immunogenicity, high antibody affinity, long-lasting immune protection and good safety;
(2) It can induce a Th1-biased immune response;
(3) The degradation of nucleic acid is avoided during the preparation process of the vaccine of the present invention. While ensuring the efficacy of the vaccine, the excessive inflammatory response caused by additional adjuvant is reduced or avoided, because no additional adjuvant is needed;
(4) The chassis particles used in the vaccine of the present invention is modified to avoid particle aggregation or precipitation, which facilitates the production of the vaccine, ensures the stability of the vaccine efficacy, and facilitates large-scale production;
(5) The vaccine structure of the present invention is convenient to adjust for the variation of the RBD antigen of the novel coronavirus, and new PLA vaccines can be produced quickly without changing the design of the chassis particles;
(6) It can provide long-term immune memory and long-lasting immune protection, providing at least about 1 year of immune protection time.

### BRIEF DESCRIPTION OF THE DRAWINGS

FIG. 1: Lysis of whole bacteria before and after induced expression of AP205 fusion protein, wherein FIG. 1A is SC-AP205 and FIG. 1B is AP205-SC.

FIG. 2: Nucleic acid gel image of SC-AP205 and AP205-SC after sucrose cushion centrifugation, wherein 1 is AP205-ST, 2 is SC-AP205, and 3 is AP205-SC.

FIG. 3: Gel image of layered collection protein of AP205 fusion protein after cesium chloride density gradient centrifugation, wherein FIG. 3A is SC-AP205, and FIG. 3B is AP205-SC.

FIG. 4: Whole bacterial lysis before and after AP205-ST induction.

FIG. 5: Gel image of layered collection protein of AP205-ST after cesium chloride density gradient centrifugation.

FIG. 6: Degradation of RBD-SC.

FIG. 7: Degradation of AP205-RBD (SC at C-terminus).

FIG. 8: Stability of the ligated product of SC-RBD and AP205-ST.

FIG. 9: The effect of the modified AP205 on the solubility of the connected product, \textcircled{1} is the unmodified wild-type AP205, and \textcircled{2} is the modified AP205 of the present invention. FIG. 9A and FIG. 9B are SDS-PAGE and nucleic acid gel images, respectively.

FIG. 10: The effect of adjusting the antigen ratio on the solubility of the connected product, FIG. 10A, FIG. 10B and FIG. 10C are SDS-PAGE, nucleic acid gel, and Coomassie R-250 images, respectively.

FIG. 11: The effect of purification condition, i.e., solution pH, on the integrity of the nucleic acid inside the VLP, FIG. 11A and FIG. 11B are SDS-PAGE and nucleic acid gel images, respectively.

FIG. 12: Changes of nucleic acids inside VLPs under different pH gradients.

FIG. 13: Anti-RBD IgG antibodies produced in mice immunized with PLA-SARS-CoV2 vaccine (primary immunization).

FIG. 14: Anti-RBD IgG antibodies produced in mice immunized with PLA-SARS-CoV2 vaccine (secondary immunization).

FIG. 15: Changes in titers of RBD IgG antibodies produced by PLA-SARS-CoV2 vaccine after primary immunization and re-immunization.

FIG. 16: Neutralizing antibodies produced in mice immunized with PLA-SARS-CoV2 vaccine.

FIG. 17: Anti-RBD IgG antibodies produced in macaques immunized with PLA-SARS-CoV2 vaccine.

FIG. 18: Neutralizing antibodies produced in macaques immunized with PLA-SARS-CoV2 vaccine.

FIG. 19: Virus load in the lung of macaques immunized with PLA-SARS-CoV2 vaccine.

FIG. 20: Th1-biased immune response induced by PLA-SARS-CoV2 vaccine.

(A) Anti-RBD of the indicated Ig isotypes were detected in serum collected after the second immunization. The columns represent different immune groups, from left to right AP205-RBD, RBD+Alum+CpG, RBD+Alum.

(B) Ratio of RBD-specific IgG2a/c titers to IgG1 titers. One-way ANOVA was used to determine significant differences among the three groups. The AP205-RBD immunized group was then compared with one of the RBD protein immunized groups using Tukey's multiple comparison test. Adjusted p-values are used to indicate statistical significance (**p<0.01, ***p<0.001).

(C) Representative data of the IFNγ ELISpot assay. IFNγ+ cells were detected in splenocytes from naive mice or mice previously immunized for 5 days with a second dose of AP205-RBD or RBD+Alum. A library of 15-mer peptides derived from RBD sequences was used to stimulate cells in vitro. Pool1 and pool2 contain peptides derived from SARS-CoV-2 S protein 420-459 and 511-549 aa, respectively. Intact RBD proteins are also used for stimulation.

(D) Quantification of IFNγ+ cells in (C). Symbols represent data collected from individual mice. Columns represent the geometric mean of each group. The number (n) of mice tested in each group is shown.

FIG. 21: Persistent humoral memory induced by PLA-SARS-CoV2 vaccine.

(A-B) Splenocytes or bone marrow (BM) cells from mice immunized with AP205-RBD twice 3-4 months previously were examined for anti-RBD IgGsecreting cells by ELISpot assay. Representative data are shown in (A) and quantification data are shown in (B). Symbols represent data collected from individual mice; columns represent geometric means for each group.

(C) Anti-RBD IgG was detected in mice immunized with AP205-RBD up to 1 year after immunization. Geometric means and standard deviations are shown for each time point. There were six to eight mice at each time point, except for the last one, which had four.

FIG. 22: Ligation assay for PLA-SARS-CoV2(delta) vaccine. (A) Coomassie brilliant blue staining. (B) EB staining.

FIG. 23: Anti-RBD and anti-RBD (delta) IgG antibodies produced in mice immunized with PLA-SARS-CoV2(delta) vaccine.

FIG. 24: Neutralization of serum immuned by PLA-SARS-CoV2 vaccine against different mutant pseudoviruses.

### DETAILED DESCRIPTION OF THE INVENTION

The S protein of SARS-CoV-2 virus contains two functional subunits, S1 and S2. S1 and S2 consist of an extracellular domain (ECD) and a single transmembrane helix, which mediate receptor binding and membrane fusion, respectively. S1, consisting of N-terminal domain (NTD) and receptor-binding domain (RBD), is critical for determining tissue tropism and host range. The S protein binds to the receptor angiotensin-converting enzyme 2 (ACE2) through RBD, mediating the virus into the host cell.

AP205 protein (hereinafter referred to as AP205) is the main capsid protein of the newly identified AP205 RNA phage, which can self-assemble into virus-like particles (VLP). The VLP can display the connected organic molecules on its surface in a repeating manner, so as to enhance the immunogenicity of the organic molecules. The SpyTag/SpyCatcher system is widely used in the field of proteins because of its ability to spontaneously form stable isopeptide bonds under various conditions, and the VLP and the displayed organic molecules can be connected through the SpyTag/SpyCatcher.

The inventors unexpectedly found that the fusion protein constructed by the phage AP205 capsid protein sequence (hereinafter referred to as the AP205 sequence) and the SpyCatcher sequence cannot be normally assembled into a virus-like particle, and only the fusion protein formed by constructing SpyTag at the C-terminus of the AP205 sequence can be assembled normally (Example 1). The inventors accidentally discovered that the modification of the AP205 sequence and the resulting difference in the particle structure can significantly improve the solubility of the final connected product (Example 3); in addition, the ratio of the two when the chassis particle is connected to the antigen would also affect the solubility of the connected product (Example 4). The present inventors found that when the pH value of the solution in the VLP purification process is too high, it would destroy the RNA in the VLP or even completely degrade it. An appropriate pH value can ensure the retention of the RNA components in the VLP particles. The RNA nucleic acid inside the VLP is one of the key factors for PLA vaccine without adjuvant to work.

The present inventors found that the antigen from SARS-Cov2 virus was connected with structurally optimized AP205 VLP through SpyTag/SpyCatcher connection. The formed pathogen-like antigen vaccine has strong immunogenicity, able to produce high antibody affinity, long-lasting immune protection and good safety. Meanwhile, the production process of the raw materials of this vaccine is mature, the cost is low and it is easy to produce on a large scale, so that it is convenient to make adjustments for the variation of virus antigens.

Therefore, in one aspect, the present invention relates to a soluble pathogen-like antigen complex comprising: (1) a virus-like particle (VLP), which is self-assembled by a first fusion protein comprising a viral capsid protein or a variant thereof at its N-terminus and SpyTag at its C-terminus, (2) a second fusion protein, comprising an antigen from S1 protein of SARS-CoV2 virus or a variant thereof and SpyCatcher, though SpyCatcher could be located at the N terminus and C terminus of the second fusion protein, preferably the SpyCatcher is at the N-terminus of the second fusion protein; wherein, the virus-like particle also encapsulates nucleic acid inside it, and wherein the virus-like particle and the antigen from the S1 protein of SARS-CoV2 virus are covalently connected through the SpyCatcher and the SpyTag, so that the antigen from S1 protein of SARS-CoV2 virus or a variant thereof is displayed on the surface of the virus-like particle.

According to the soluble pathogen-like antigen complex of the present invention, wherein the nucleic acid encapsulated in the virus-like particle is the nucleic acid which is encapsulated by the virus-like particle when self-assembling and is from host bacteria used to express the virus-like particles, preferably the host bacteria is Escherichia coli, and preferably the nucleic acid is RNA.

The soluble pathogen-like antigen complex of the present invention, wherein the capsid protein is from Escherichia coli phage Q3, MS2 or AP205, preferably from Escherichia coli phage AP205.

The soluble pathogen-like antigen complex of the present invention, wherein the sequence of the capsid protein of the phage AP205 has at least 90%, 91%, 92%, 93%, 94%, 95%, 96%, 97%, 98%, 99% or 100% identity with SEQ ID NO: 1.

The soluble pathogen-like antigen complex of the present invention, wherein the sequence of the capsid protein of the phage AP205 is SEQ ID NO: 1.

The soluble pathogen-like antigen complex of the present invention, wherein the antigen is a RBD sequence of the S1 protein of SARS-CoV2 virus.

The soluble pathogen-like antigen complex of the present invention, wherein the sequence of the antigen has at least 90%, 91%, 92%, 93%, 94%, 95%, 96%, 97% %, 98%, 99% or 100% identity with SEQ ID NO: 2.

The soluble pathogen-like antigen complex of the present invention, wherein the sequence of the antigen is SEQ ID NO: 2.

The soluble pathogen-like antigen complex of the present invention, wherein the sequence of the SpyTag has at least 90%, 91%, 92%, 93%, 94%, 95%, 96%, 97%, 98%, 99% or 100% identity with of SEQ ID NO: 3, and the sequence of the SpyCatcher has at least 90%, 91%, 92%, 93%, 94%, 95%, 96%, 97%, 98%, 99% or 100% identity with SEQ ID NO: 4.

The soluble pathogen-like antigen complex of the present invention, wherein in the first fusion protein, the phage capsid protein or a variant thereof is linked with the SpyTag via a first linker peptide, and in the second fusion protein, the antigen or a variant thereof is linked to the SpyCatcher via a second linker peptide.

The soluble pathogen-like antigen complex of the present invention, wherein the sequence of the first linker peptide is SEQ ID NO: 5, and the sequence of the second linker peptide is SEQ ID NO: 6.

The present invention also relates to a nucleic acid sequence or a nucleic acid molecule encoding the first fusion protein and the second fusion protein and a vector comprising the nucleic acid sequence or nucleic acid molecule.

In some embodiments, the polynucleotides or nucleic acid molecules or vectors described herein can be codon optimized.

In some embodiments, a polynucleotide or nucleic acid molecule or vector described herein may be a degenerate version thereof.

The soluble pathogen-like antigen complex of the present invention, wherein the second fusion protein is connected to the virus-like particle at a ratio of less than or equal to 1:1, preferably connected at a ratio of 1:6-1:12, and preferably connected at a ratio of 1:6, 1:7, 1:8, 1:9, 1:10, so as to ensure the solubility and immunogenicity of the pathogen-like antigen complex, the ratio is calculated as the ratio of the SpyCatcher on the second fusion protein to the SpyTag on the virus-like particle.

Another aspect of the present invention relates to a pathogen-like antigen vaccine composition, comprising the soluble pathogen-like antigen complex according to the present invention and pharmaceutically acceptable carriers and/or excipients.

A further aspect of the present invention relates to a method for preventing and/or treating diseases associated with SARS-CoV2 virus infection in a subject in need thereof, wherein a preventive and/or therapeutically effective amount of the soluble pathogen-like antigen complex or vaccine composition of the present invention is administered to the subject.

In some embodiments, the associated diseases could be caused by SARS-COV-2 virus and/or its mutants.

In some embodiments, the associated disease could be COVID-19.

The term “fusion protein” used herein refers to a genetically engineered protein encoded by a nucleotide sequence formed by two or more complete or partial genes or a series of nucleic acids combined together. Alternatively, fusion proteins can be made by combining two or more heterologous peptides.

The term “linker peptide” as used herein means one or more (e.g., about 2-10) amino acid residues between two adjacent motifs, regions or domains of a polypeptide, such as between the antigenic peptide or between the antigenic peptide and an adjacent peptide encoded by the multiple translation leading sequence, or between the antigenic peptide and a spacer or cleavage site. Linker peptides can be derived from the construct design of the fusion protein (e.g., amino acid residues resulting from the use of restriction enzyme sites during construction of a nucleic acid molecule encoding the fusion protein).

The term “variant” as used herein refers to a protein or nucleic acid molecule whose sequence is similar but not identical to a reference sequence, wherein the activity of the variant protein (or the protein encoded by the variant nucleic acid molecule) is not significantly altered. These variations in sequence may be naturally occurring variations or may be engineered using genetic engineering techniques known to those skilled in the art. Examples of such techniques can be found in Sambrook J, Fritsch E F, Maniatis T et al., in Molecular Cloning-A Laboratory Manual, 2nd Edition, Cold Spring Harbor Laboratory Press, 1989, pp. 9.31-9.57), or Current Protocols in Molecular Biology, John Wiley & Sons, N.Y. (1989), 6.3.1-6.3.6. With regard to variants, any type of change in amino acid or nucleic acid sequence is permissible as long as the activity of the resulting variant protein or polynucleotide is not significantly altered. Examples of such variations include, but are not limited to, deletions, insertions, substitutions, and combinations thereof. According to their properties, amino acids can be classified into charged amino acids, uncharged amino acids, polar uncharged amino acids, and hydrophobic amino acids. Thus, protein variants containing substitutions may be those in which amino acids are substituted by amino acids from the same group. Such substitutions are known as “conservative” substitutions.

As used herein, the term “antigen” or variants thereof means a polypeptide that can stimulate a cell to generate an immune response.

As used herein, the term “virus-like particles (VLPs)” is a particle assembled from one or more viral structural proteins, which has a similar external structure and antigenicity to viral particles, but does not contain viral genes.

The terms “vaccine” and “vaccine composition” used in the present invention refer to a pharmaceutical composition containing corresponding virus antigens, which can induce, stimulate or enhance the subject's immune response against the corresponding virus.

The term “nucleic acid” or “nucleic acid molecule” as used herein means any one of deoxyribonucleic acid (DNA), ribonucleic acid (RNA), oligonucleotides, fragments produced, for example, by polymerase chain reaction (PCR) or by in vitro translation, and fragments produced by any one or more of ligation, cleavage, endonuclease action, or exonuclease action. In certain embodiments, nucleic acids of the disclosure are produced by PCR. Nucleic acids can be composed of monomers that are naturally occurring nucleotides (such as deoxyribonucleotides and ribonucleotides), analogs of naturally occurring nucleotides (e.g., naturally occurring nucleotides in α-enantiomeric forms) or combinations thereof. Modified nucleotides may have modifications in or in place of sugar moieties, or pyrimidine or purine base moieties.

The term “construct” as used herein means any polynucleotide comprising a recombinant nucleic acid. The construct can be present in a vector (e.g., bacterial vector, viral vector), or can be integrated into the genome. A “vector” is a nucleic acid molecule capable of transporting another nucleic acid. A vector can be, for example, a plasmid, cosmid, virus, RNA vector or linear or circular DNA or RNA molecule, which can include chromosomal, non-chromosomal, semi-synthetic or synthetic nucleic acids. Exemplary vectors are those capable of autonomous replication (episomal vectors) and/or expression of nucleic acids to which they are linked (expression vectors).

As used herein, the terms “signal peptide” and “leading sequence” are used interchangeably herein and refer to an amino acid sequence that can be linked to the amino terminus of the proteins set forth herein. A signal peptide/leading sequence usually directs the localization of the protein. The signal peptide/leading sequence used herein preferably facilitates secretion of the protein from the cell in which it is produced. The signal peptide/leading sequence is often cleaved from the rest of the protein (often called the mature protein) after secretion from the cell. A signal peptide/leading sequence is attached to the N-terminus of the protein and is about 9 to 200 nucleotides (3 to 60 amino acids) in length. The signal peptide used in the present invention can be the signal peptide sequence of S protein of SARS-COV-2 virus or the signal peptide sequence from other eukaryotic/viral proteins.

The term “expression vector” as used herein refers to a DNA construct comprising a nucleic acid molecule operably linked to suitable control sequences enabling expression of the nucleic acid molecule in a suitable host. Such control sequences include a promoter for effecting transcription, optionally an operator sequence for controlling such transcription, a sequence encoding a suitable mRNA ribosomal binding site, and sequences controlling termination of transcription and translation. The vector can be a plasmid, phage particle, virus, or simply a potential genomic insert. Viral vectors can be DNA-based (eg, adenovirus or vaccinia virus) or RNA-based, including oncolytic viral vectors (eg, VSV), replication competent or non-replicative. Once transformed into a suitable host, the vector can replicate and function independently of the host genome, or, in some cases, can integrate into the genome itself. In this specification, “plasmid”, “expression plasmid”, and “vector” are often used interchangeably.

The term “expression” as used herein means the process of producing a polypeptide based on the nucleic acid sequence of a gene. The processes include transcription and translation. Translation can start at an unconventional start codon, such as the CUG codon, or translation can start at several start codons (standard AUG and unconventional) to produce more protein than mRNA produced (on a per mole basis quantity).

As used herein, the term “introducing” in the context of inserting a nucleic acid sequence into a cell means “transfection” or “transformation” or “transduction” and includes the integration of a nucleic acid sequence into a eukaryotic or prokaryotic cell, wherein the nucleic acid sequence can be integrated into the genome of the cell (e.g., chromosomal, plasmid, plastid, or mitochondrial DNA), transformed into an autonomous replicon, or expressed transiently (eg, transfected mRNA).

Recombinant methods for expressing exogenous or heterologous nucleic acids in cells are well known in the art. Such methods can be found, for example, in Sambrook et al., Molecular Cloning: A Laboratory Manual, Third Edition, Cold Spring Harbor Laboratory, New York (2001); and Ausubel et al., Current Protocols in Molecular Biology, John Wiley and Sons, Baltimore, MD (1999). Genetic modification of a nucleic acid molecule encoding a fusion antigen protein can confer biochemical or metabolic capabilities on a recombinant or non-natural cell altered from its naturally occurring state.

The term “host” as used herein refers to any organism or cell thereof into which the construct of the invention can be introduced, whether eukaryotic or prokaryotic, in particular, a host in which RNA silencing occurs. In specific embodiments, “hosts” include Escherichia coli such as E. coli. The term “host” refers to eukaryotes, including unicellular eukaryotes such as yeast and fungi and multicellular eukaryotes such as animals. Non-limiting examples include invertebrates (e.g., insects, coelenterates, echinoderms, nematodes etc.); eukaryotic parasites (e.g., malaria parasites such as Plasmodium falciparum, worms, etc.); vertebrates (e.g., fish, amphibians, reptiles, birds, mammals); and mammals (e.g., rodents, primates such as humans and non-human primates). Thus, the term “host cell” properly encompasses cells of such eukaryotes as well as cell lines derived from such eukaryotes.

The term “adjuvant” used herein refers to a natural or synthetic substance that promotes the body's T cell or B cell response by enhancing the activity of macrophages and participates in the immune response to haptens or antigens.

The term “prevention and/or treatment” as used herein refers to inhibiting the replication, spread or colonization of the corresponding virus in the host, as well as alleviating the symptoms of the virus-infected disease or disorder. The treatment is considered to be therapeutically effective if there is a reduction in virus load, a reduction in symptoms and/or an increase in food intake and/or growth.

“Therapeutically effective amount (or dose)” or “effective amount (or dose)” of a compound or composition as used herein means the amount of the compound sufficient to cause improvement of one or more symptoms of the disease being treated in a statistically significant manner. The precise amount depends on numerous factors, e.g., the activity of the composition, the method of delivery employed, the immunostimulatory capacity of the composition, the intended patient and patient considerations, etc., and can be readily determined by one of ordinary skill in the art. A therapeutic effect may include, directly or indirectly, the alleviation of one or more symptoms of a disease, and a therapeutic effect may also include, directly or indirectly, the stimulation of a cellular immune response.

As used herein, the term “pharmaceutically acceptable carrier” includes any carrier that does not, by itself, induce the production of antibodies deleterious to the individual receiving the pharmaceutical composition. Suitable carriers are usually large, slowly metabolized macromolecules, such as proteins, polysaccharides, polylactic acid, polyglycolic acid, amino acid polymers, amino acid copolymers, lipid aggregates (such as oil droplets or liposomes), and the like. These pharmaceutically acceptable carriers are well known to those of ordinary skill in the art.

The term “subject” as used herein may be any organism capable of mounting a cellular immune response, such as a human, pet, livestock, exhibit animal, zoo specimen, or other animal. For example, a subject can be a human, non-human primate, dog, cat, rabbit, rat, mouse, guinea pig, horse, cow, sheep, goat, pig, and the like. Subjects who need to be administered a therapeutic agent as described herein include those who have been infected by SARS-COV-2 virus or even have developed a virus infection-related disease, or are at risk of SARS-COV-2 virus infection.

The term “subject in need” as used herein means a subject at high risk of or suffering from a disease, disorder or condition. The disease, disorder, or condition is suitable for treatment or improvement with the compound or combination provided herein. In some embodiments, the subject in need is a human.

For complexes comprising pathogen-like antigens as described herein, the desired outcome is a safe product capable of inducing durable protective immunity with minimal side effects. Compared to other strategies (e.g., whole live or attenuated pathogens), being inexpensive to produce could minimize or eliminate contraindications that have otherwise been (generally) associated with the use of whole or attenuated viral immune compositions. The ability to respond rapidly to infectious disease emergencies is one benefit of the effective application of the embodiments disclosed herein, whether in the context of biodefense or immunotherapy or technology.

The pathogen-like antigen vaccines of the present invention can be administered by, for example, intramuscular injection, subcutaneously, intranasally, transmucosally presented, intravenously or by intradermal or subcutaneous administration.

The following will provide examples of the specific embodiments for the invention of pathogenic antigen vaccines. However, it should be understood that these examples do not in any way limit the scope of the present invention.

### EXAMPLES

As an adjuvant-free protein engineering vaccine, the novel coronavirus pathogen-like antigen (PLA) vaccine of the present invention has sufficient structural stability, is soluble, and would not aggregate or precipitate. The nucleic acids present inside the VLP, acting as a TLR stimulator, ensure that the vaccine is sufficiently immunogenic. Compared with traditional vaccines that require the addition of adjuvants, the PLA-SARS-CoV2 vaccine of the present invention could induce significantly higher levels of specific antibodies and neutralizing antibodies and has significantly higher efficacy compared to traditional vaccines in the challenge test.

### Example 1: The Effect of the Linking Format of SpyCatcher (SC) and SpyTag (ST) with AP205 on VLP Self-Assembly

### (1) Construction of Fusion Protein SC-AP205 (SC is Located at the N-Terminus of AP205) Expression Plasmid

The amino acid sequence of SC is SEQ ID NO: 4. The amino acid sequence of modified AP205 (non-wild type) is SEQ ID NO: 1. The two are linked by a linker sequence SEQ ID NO: 5.

### I. Construction of AP205 Expression Vector

A full-length 393 bp cDNA (SEQ ID NO: 9) fragment encoding AP205 was artificially synthesized. A BamHI restriction site was added at its 5′ end and a GSGGSG linker, an AgeI restriction site, a stop codon TAA, and a KpnI restriction site were added at its 3′ end. The synthesized AP205 cDNA fragment (1 μg) and pET21 plasmid (1 μg) were digested with BamHI (Takara 1010A) and KpnI endonuclease (Takara 1068A) at 37° C. for 2 hours, respectively. The digested cDNA fragment and pET21a plasmid fragment were then separated by agarose gel electrophoresis. The isolated cDNA fragment and pET21a plasmid fragment were purified using a small amount of DNA product purification kit (Zhuangmeng Bio ZP201-3). The purified cDNA fragment was further subjected to DNA ligation reaction with the pET21a plasmid fragment to construct a pET21a plasmid containing the cDNA fragment (called pET21a-AP205 plasmid). The ligase was T4 DNA ligase (Takara 2011A), the ligation buffer was T4 DNA Ligase Buffer (Takara 2011A), the ratio of the pET21a plasmid fragment to the AP205 cDNA fragment in the ligation reaction was about 1:3, and the total DNA was about 200 ng. Ligation was done at 22° C. for 2 hours. The pET21a-AP205 plasmid was transformed into the expression host as follows: 15 μl of the ligation reaction solution was added to 150 μl of XLI-Blue competent E. coli (Transgene CD401-02) at 42° C. for 1 minute. 150 μl was pipetted onto an ampicillin-resistant LB plate and the plated was incubated at 37° C. for 14-16 hours. A single colony was taken from the plate, and the plasmid DNA was extracted with a plasmid purification kit (Transgene EM101-02) and verified by enzyme digestion, confirming the successful construction of the pET21a-AP205 plasmid.

### II. Construction of SC-AP205 Expression Vector

A full-length 276 bp cDNA fragment encoding SC (SEQ ID NO: 10) was artificially synthesized by PCR (upstream primer (SEQ ID NO: 13): acgggatccATGTCGTACTACCATCACCATC, downstream primer (SEQ ID NO: 14): cccggatccactgccgctacctccAATATGAGCGTCACCTTTAGTTGC, the PCR program is \textcircled{1} 94° C. for 5 minutes \textcircled{2} 94° C. for 30 seconds \textcircled{3} 58° C. for 30 seconds \textcircled{4} 72° C. for 1 minute, \textcircled{2} \textcircled{3} \textcircled{4} for 30 cycles, \textcircled{5} 72° C. for 5 minutes \textcircled{6} keeping at 4° C.). BamHI restriction sites were added at both its 5′ and 3′ ends. The synthesized SC cDNA fragment (1 μg) and pET21a-AP205 plasmid (1 μg) were digested with BamHI endonuclease (Takara 1010A) at 37° C. for 2 hours, respectively. The digested cDNA fragment and pET21a-AP205 plasmid fragment were then separated by agarose gel electrophoresis. The isolated cDNA fragment and the pET21a-AP205 plasmid fragment were purified using a small DNA product purification kit (Zhuangmeng Bio ZP201-3). The purified cDNA fragment was further subjected to DNA ligation reaction with the pET21a-AP205 plasmid fragment to construct the pET21a-AP205 plasmid containing the cDNA fragment (called pET21a-SC-AP205 plasmid). The ligase was T4 DNA ligase (Takara 2011A), the ligation buffer was T4 DNA Ligase Buffer (Takara 2011A), the ratio of the pET21a-AP205 plasmid fragment to the SC cDNA fragment in the ligation reaction was about 1:3, and the total DNA was about 200 ng. Ligation was done at 22° C. for 2 hours. The pET21a-SC-AP205 plasmid was transformed into the expression host as follows: 15 μl of the ligation reaction solution was added to 150 μl of XLI-Blue competent E. coli (Transgene CD401-02) at 42° C. for 1 minute. 150 μl was pipetted onto an ampicillin-resistant LB plate and the plate was incubated at 37° C. for 14-16 hours. A single colony was taken from the plate, and the plasmid DNA was extracted with a plasmid purification kit (Transgene EM101-02) and verified by enzyme digestion, confirming the successful construction of the pET21a-SC-AP205 plasmid.

### (2) Construction of Fusion Protein AP205-SC (SC is Located at the C-Terminus of AP205) Expression Plasmid

The fusion protein AP205-SC expression plasmid was prepared in the same way as in above (1), except that a full-length 276 bp SC cDNA fragment was artificially synthesized by PCR (upstream primer: acgaccggtATGTCGTACTACCATCACCATC (SEQ ID NO: 15), downstream primer: cccaccggtAATATGAGCGTCACCTTTAGTTGC (SEQ ID NO: 16), the PCR program is \textcircled{1} 94° C. for 5 minutes, \textcircled{2} 94° C. for 30 seconds, \textcircled{3} 58° C. for 30 seconds, \textcircled{4} 72° C. for 1 minute, \textcircled{2} \textcircled{3} \textcircled{4} for 30 cycles, \textcircled{5} 72° C. for 5 minutes, and \textcircled{6} keeping at 4° C.). AgeI restriction sites were added at both its 5′ and 3′ ends. The synthesized SC cDNA fragment (1 μg) and pET21a-AP205 plasmid (1 μg) were digested with AgeI endonuclease (NEB R0552V) and ligated to construct pET21a-AP205-SC plasmid.

### (3) Construction of Fusion Protein AP205-ST (ST is Located at the C-Terminus of AP205 Sequence) Expression Plasmid

The fusion protein AP205-ST expression plasmid was constructed in the same way as in above (1), except that the ST encoding DNA sequence (gcccacatcgtgatggtggacgcctacaagccgacgaag) was synthesized through the following process (the encoded amino acid sequence is SEQ ID NO: 4):

Artificially synthesized primers:

F:
(SEQ ID NO: 17)
ccggtggtagcggcgcccacatcgtgatggtggacgcctacaagccgac
gaaga
R:
(SEQ ID NO: 18)
ccggtcttcgtcggcttgtaggcgtccaccatcacgatgtgggcgccgc
tacca

The DNA sequence encoding ST was obtained by annealing PCR (5 μl 200 μM Primer-F, 5 μl 200 μM Primer-R, 2 μl 10× annealing buffer (100 mM Tris 8.0, 1 M NaCl, 10 mM EDTA), 8 μl dH₂O; the PCR program was set to be at 99° C. for 3 min, and the temperature was decreased by 0.5° C. every 30 seconds for 99-20° C., and finally kept at 4° C.). The pET21a-AP205 plasmid (1 μg) was digested with AgeI endonuclease (NEB R0552V) at 37° C. for 2 hours. The digested pET21a-AP205 plasmid fragment was then separated by agarose gel electrophoresis. The isolated pET21a-AP205 plasmid fragment was purified using a small amount of DNA product purification kit (Zhuangmeng Bio ZP201-3). The ST DNA fragment obtained by PCR was further subjected to DNA ligation reaction with the purified pET21a-AP205 plasmid fragment to construct a pET21a-AP205 plasmid containing the DNA fragment (called pET21a-AP205-ST plasmid). The ligase was T4 DNA ligase (Takara 2011A), the ligation buffer was T4 DNA Ligase Buffer (Takara 2011A), the ratio of pET21a-AP205 plasmid fragment to ST DNA fragment in the ligation reaction was about 1:3, and the total DNA was about 200 ng. Ligation was done at 22° C. for 2 hours. The pET21a-AP205-ST plasmid was transformed into the expression host as follows: 15 μl of the ligation reaction solution was added to 150 μl of XLI-Blue competent E. coli (full gold CD401-02) at 42° C. for 1 minute. 150 μl was pipetted onto an ampicillin-resistant LB plate and the plate was incubated at 37° C. for 14-16 hours. Plasmid DNA was extracted with a plasmid purification kit (Transgene EM101-02) and verified by enzyme digestion, confirming that the pET21a-AP205-ST plasmid was successfully constructed.

(4) Fusion Protein Expression and Self-Assembly into VLP, Purification of VLP

Fusion protein expression: the BL21 (DE3) competent E. coli (CD601-02) that has been verified by sequencing to be transformed with the previously constructed plasmid was used. A single clone was picked into ampicillin-resistant LB medium and and subject to shaking overnight at 37° C., 220 rpm. On the second day, the culture was expanded, and the inducer IPTG (Yisheng Biology 10902ES08) at a final concentration of 0.1 mM was added to induce the expression of the fusion protein when the OD value in the logarithmic growth phase was 0.6-0.9, and the bacteria was harvested after 5 hours of induction.

Purification: the harvested E. coli (6000 rpm for 10 minutes) was centrifuged to obtain cell pellets. The pellet was resuspended in 20 mM Tris pH 7.5. The bacteria were sonicated to obtain the lysed supernatant. The supernatant was centrifuged twice (5000 rpm for 10 minutes, 20000 g for 30 minutes) to remove insoluble impurities such as cell debris, and then particle proteins were centrifuged through a 30% sucrose cushion (in a 12 ml centrifuge tube, 2 ml 30% sucrose was added at the bottom, and 10 ml lysate supernatant was added on it, 33000 rpm for 3.5 hours). The particle proteins were resuspended with 1 ml PBS (KCl 2.6 mM, KH₂PO₄ 1.47 mM, NaCl 136 mM, Na₂HPO₄·12H₂O 8 mM), and then the impurity proteins were separated from the target protein by cesium chloride density gradient centrifugation (in a 5 ml ultracentrifuge tube, 2 ml of 50% cesium chloride was added, 2 ml of 24% cesium chloride was added, and finally 1 ml of sample was added) (200000 g, 22 hours). Samples in layers were collected and a protein gel was run to confirm the location of the target fusion protein. The corresponding layer of protein was taken and dialyzed in PBS for storage.

### (5) Results

SC-AP205, AP205-SC and AP205-ST expression plasmids can express the corresponding fusion protein well.

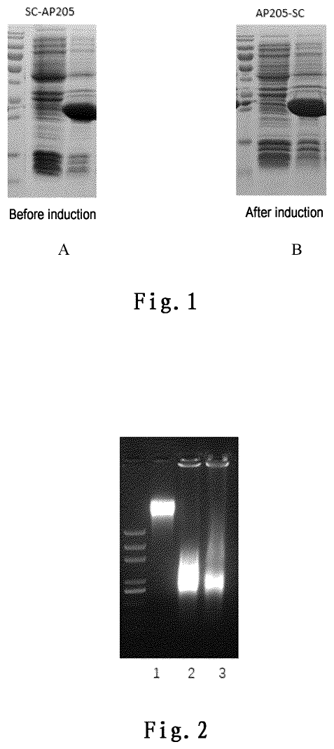

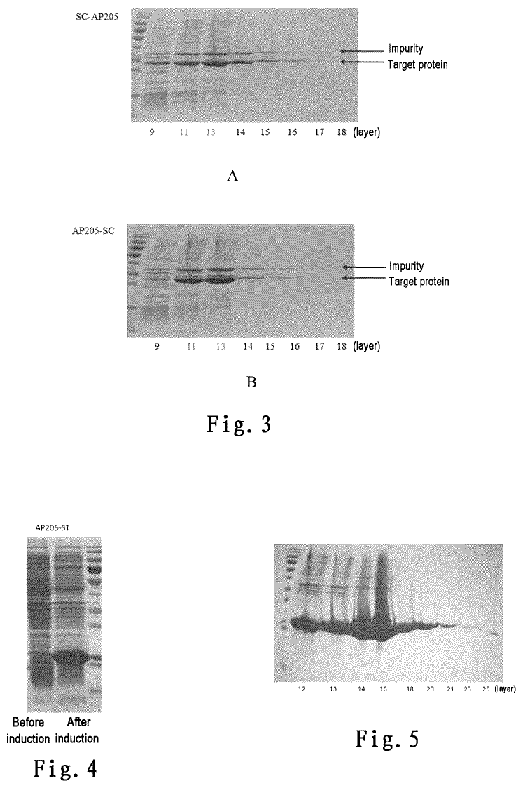

The comparison protein gel image of the whole bacterial lysate before and after induction of SC-AP205 and AP205-SC expression plasmids is shown in FIG. 1, showing that the target fusion protein was expressed. However, when purifying SC-AP205 and AP205-SC, the pellet at the bottom of the centrifuge tube was difficult to be resuspended after sucrose cushion centrifugation, indicating that there was a problem with the self-assembly of SC-AP205 and AP205-SC, and well-dispersed, non-aggregated VLPs could not be obtained (see FIG. 2). Samples in layers were collected after cesium chloride density gradient centrifugation. It was visibly turbid to the naked eye before the 13th layer, and it was clear after the 14th layer. However, from the protein gel band, the target protein could not be separated from the impurity protein, and the yield was very small (see FIG. 3).

For the AP205-ST expression plasmid, VLPs that were successfully self-assembled from AP205-ST were obtained in the subsequent purification (see FIGS. 2 and 4). Obvious target fusion protein bands could be seen in the 14th to 20th layers in the protein gel image of layered collection protein after the cesium chloride density gradient centrifugation (see FIG. 5). The corresponding layer was taken and dialyzed into PBS to obtain purified, well-dispersed, and non-aggregated VLPs assembled from AP205-ST (AP205-ST VLPs). In the laboratory stage, 50-60 mg VLP could be obtained per liter of bacteria.

### Example 2: The Effect of the Connecting Format of SpyCatcher (SC) and Antigen on the Stability of PLA

### (1) Construction of Fusion Protein RBD-SC (SC is Located at the C-Terminus of RBD) Expression Plasmid

The full-length 1068 bp cDNA (SEQ ID NO: 11) fragment encoding RBD-SC was artificially synthesized, and the Kozak sequence GCCACC for regulating protein expression and KpnI restriction site were added to its 5′ end and XhoI restriction site was added to the 3′ end. The synthesized RBD-SC cDNA fragment (1 μg) and pCEP4 plasmid (1 μg) were digested with KpnI and XhoI endonucleases (Takara) at 37° C. for 2 hours, respectively. The digested cDNA fragment and pCEP4 plasmid fragment were then separated by agarose gel electrophoresis. The isolated cDNA fragments and pCEP4 plasmid fragments were purified using a small amount of DNA product purification kit (Zhuangmeng Bio ZP201-3). The purified cDNA fragment was further subjected to DNA ligation reaction with the pCEP4 plasmid fragment to construct a pCEP4 plasmid containing the cDNA fragment (called pCEP4-RBD-SC plasmid). The ligase was T4 DNA ligase (Takara 2011A), the ligation buffer was T4 DNA Ligase Buffer (Takara 2011A), the ratio of the pCEP4 plasmid fragment to the RBD-SC cDNA fragment in the ligation reaction was about 1:3, and the total DNA was about 200 ng. Ligation was done at 22° C. for 2 hours. The pCEP4-RBD-SC plasmid was transformed into the expression host as follows: 15 μl of the ligation reaction solution was added to 150 μl of XLI-Blue competent E. coli (Transgene CD401-02) at 42° C. for 1 minute. 150 μl was pipetted onto an ampicillin-resistant LB plate and the plated was incubated at 37° C. for 14-16 hours. A single colony was taken from the plate, and the plasmid DNA was extracted with a plasmid purification kit (Transgene EM101-02) and verified by enzyme digestion, confirming the successful construction of the pCEP4-RBD-SC plasmid.

The pCEP4-RBD-SC plasmid was extracted from the host bacteria using the endotoxin-free large-scale extraction kit (Tiangen DP117). The extracted pCEP4-RBD-SC plasmid was transfected into 293F cell line (Life technologies) using PEI reagent (polyscience 23966-1). The transfection mixture was prepared: \textcircled{1} 300 μg of plasmid plus 15 ml of SMM 293-TII medium (Sino biological M293TII), \textcircled{2} 1.5 ml of PEI plus 15 ml of SMM 293-TII medium. The two were mixed and kept at room temperature for 2 minutes. The transfection mixture \textcircled{1} \textcircled{2} were mixed fully and kept at room temperature for 15 minutes, followed by adding the transfection mixture to 300 ml of cell fluid with a cell density of 2×10⁵ cells/ml. They were mixed well and kept in a shaker at 37° C., 5% carbon dioxide, and 125 rpm. In the middle, 7 ml feed SMS 293-SUPI (Sino biological M293-SUPI) was added every two days, and the cells were harvested on the seventh day.

Insoluble impurities such as cell debris were removed by two-step centrifugation (500 g for 10 minutes, 8000 rpm for 30 minutes). The supernatant was passed through a 0.2 pm filter membrane to further remove insoluble impurities. Ni-NTA prepacked gravity column (BBI C600791-0005) was used to purify the expressed target protein. The steps are as follows:
a. Equilibration: it was first washed with 50 ml ultrapure water, and then 50 ml binding buffer (5 mM imidazole, 500 mM sodium chloride, 20 mM Tris, 10% glycerol, pH7.9) was used.
b. Sample loading: the cell supernatant was passed through the nickel column, and the sample loading was repeated for three times.
c. Elution: first, 50 ml of washing buffer (30 mM imidazole, 500 mM sodium chloride, 20 mM Tris, 10% glycerol, pH 7.9) was used to wash away impurities. The target protein was then eluted with 50 ml of elution buffer (250 mM imidazole, 500 mM sodium chloride, 20 mM Tris, 10% glycerol, pH 7.9). The target protein solution was concentrated to about 5 ml and then dialyzed into PBS for storage.

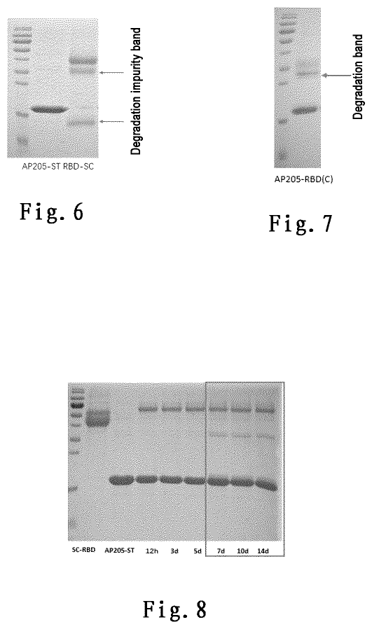

The obtained fusion protein RBD-SC had poor stability and was severely degraded when placed at 4° C. for three days (so the situation after three days was not shown in FIG. 6) (see FIG. 6).

### (2) Connection Between RBD-SC and AP205-ST VLP

RBD-SC and AP205-ST VLP at a ratio of 1:10 (one VLP is self-assembled from 180 AP205-ST sequences, i.e., there are 180 STs. This ratio refers to the ratio of the SC on the RBD and the ST on the VLP to be connected, the same below) was incubated in PBS buffer at 4° C. for 1 hour, whereby the Asp at the 7th position of the ST amino acid sequence and the Lys at the 31st position of the SC amino acid sequence spontaneously formed an isopeptide covalent bond, allowing RBD-SC to covalently connected to AP205-ST VLPs. This reaction process does not require any special enzymes and buffer systems.

The resulting connected product AP205-ST VLP/RBD-SC was completely degraded when left at 4° C. for 9 days (see FIG. 7).

### (3) Construction of Fusion Protein SC-RBD (SC is Located at the N-Terminus of RBD) Expression Plasmid and Connection of SC-RBD and AP205-ST VLP

The same method as above (1) was used to construct fusion protein SC-RBD expression plasmid, and the same method as above (2) was used to connect SC-RBD and AP205-ST VLP. The fusion protein SC-RBD sequence is SEQ ID NO: 8. The full-length 1059 bp SC-RBD cDNA (SEQ ID NO: 12) fragment was artificially synthesized, and the Kozak sequence GCCACC for regulating protein expression and HindIII restriction sites were added to its 5′ end and the XhoI restriction site was added to the 3′ end. The synthesized SC-RBD cDNA fragment (1 μg) and pCEP4 plasmid (1 μg) were digested with HindIII and XhoI endonucleases (Takara) at 37° C. for 2 hours, respectively.

Compared with the afore-mentioned fusion protein RBD-SC, the stability of the obtained fusion protein SC-RBD was obviously improved. Moreover, at 4° C., when the connection ratio of SC-RBD and AP205-ST VLP was 1:10, the connection product AP205-ST VLP/SC-RBD was stable within 5 days, and a small amount of antigen shedding did not occur until the 7th day. At the 14th days, most of them were still complete connected products (see FIG. 8). It could be seen that the stability of AP205-ST VLP/SC-RBD was significantly better than that of AP205-ST VLP/RBD-SC which was completely degraded on the 9th day under the same conditions.

### Example 3: Effect of the Sequence of AP205 on the Solubility of PLA

To study the effect of the sequence of AP205 on PLA solubility and the ability of AP205-ST VLP to carry external antigens, the inventors used the modified AP205 capsid protein sequence used in the present invention (i.e., five amino acids MEFGS were added to the N terminus of the wild-type (WT) AP205 capsid protein sequence. Unless otherwise specified, the AP205 used herein and the corresponding VLP and vaccine products were all prepared using the modified AP205) and the unmodified WT AP205 capsid protein sequence to perform a series of comparative experiments.

WTAP205-ST VLPs were obtained in the same manner as described above. Then it was connected with the fusion protein SC-RBD in the same way as the afore-mentioned connection method to obtain the corresponding connected product. The connected product was reduced and denatured and then subjected to SDS-PAGE to show the covalent connection between the antigen and VLP; and nucleic acid gel electrophoresis was run to detect the solubility of the connected product. The specific measurement process and conditions are as follows: the loading amount was 10 μg PLA or connection product, 1% nucleic acid gel, 90 volts, 20 minutes.

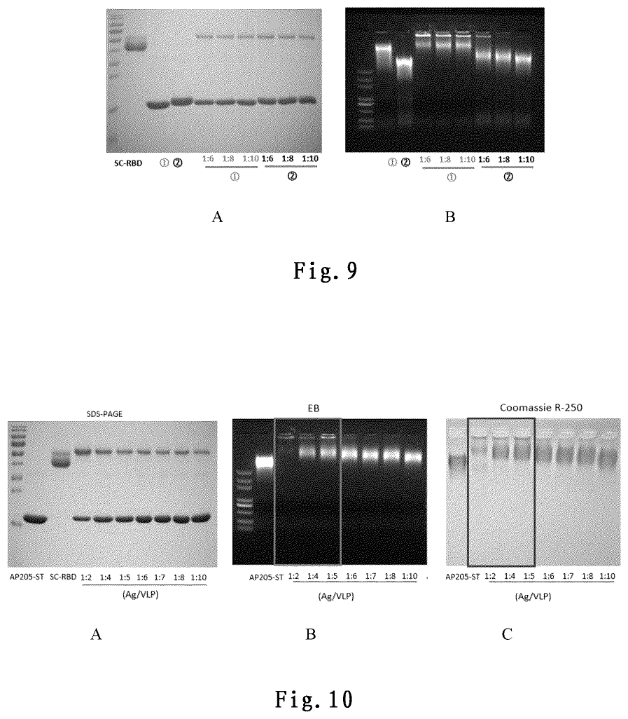

It was found that: SC-RBD could be well connected with wild-type and modified AP205-ST VLPs at different ratios (1:6, 1:8, 1:10) (see FIG. 9A). The connected products formed by WT AP205—ST VLPs were easy to aggregate, forming visible precipitates, which can be seen in the nucleic acid gel holes; while the solubility of the connected products formed by the modified AP205-ST VLPs was significantly improved, and there was no obvious visible precipitation, especially in 1:8 and 1:10 ratios (see FIG. 9B, in which \textcircled{1} was before modification and \textcircled{2} was after modification).

### Example 4: Effect of the Ratio of Antigen to VLP on PLA Solubility

The present inventors studied the effect of the ratio of antigen to VLP on the solubility of connected product PLA, to further improve the solubility of the connected product by adjusting the ratio. The inventor used SC-RBD and AP205-ST VLP to test the ratios of 1:2, 1:4, 1:5, 1:6, 1:7, 1:8, and 1:10 respectively, and the test method was the same as that in Example 3. FIG. 10A shows that connected products could be successfully obtained under these ratios; FIG. 10B shows that when the ratio of antigen to VLP was higher (1:2, 1:4, 1:5), obvious deposition could be seen in the nucleic acid gel hole, and there was obvious precipitation visible to the naked eye, indicating that there was PLA aggregation at this time. However, when the connection ratio between antigen and VLP was reduced, for example, when the connection ratio was 1:6, 1:7, 1:8, and 1:10, there was basically no visible precipitation, and there was no protein deposited in the nucleic acid gel hole. FIG. 10C is the protein staining result of the agarose gel, which shows the movement of RNA and AP205 protein in the electrophoresis. It can also be seen that there was EB fluorescence and protein staining appeared in the gel hole when the connection ratio was high, indicating PLA aggregation. It could be seen that reducing the ratio of antigen to VLP can significantly improve the solubility of the connected product. From the above experimental results, it could be seen that the connection ratio suitable for the novel coronavirus antigen RBD is about 1:6-1:12, such as 1:6, 1:7, 1:8, 1:9, 1:10.

### Example 5: Effect of VLP Purification Conditions on the Presence or Absence of RNA Inside it

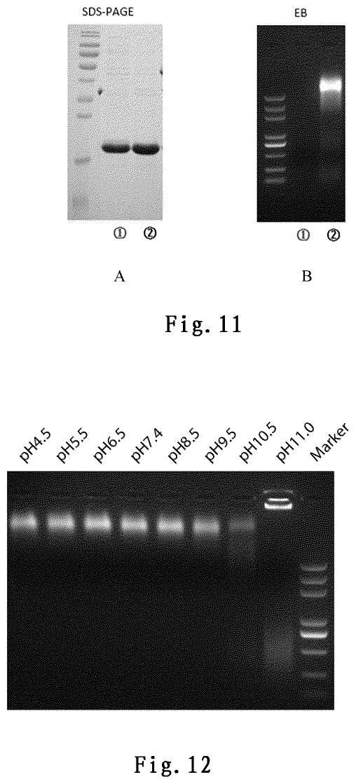

When exploring the industrial purification process of VLP, the inventors found that the RNA inside the purified VLP disappeared when the pH of the ion exchange solution was 10.5 (see FIG. 11), suggesting that the pH of the solution may affect the presence of RNA inside the VLP. Therefore, based on the VLP purification conditions described in Example 1, the inventors examined the effect of solution pH on the presence of RNA inside VLP. The specific method is as follows: adjusting the pH of PBS with hydrochloric acid and NaOH respectively, then placing 2.5 μg of purified VLP in a 37° C. water bath for 2 hours, and then detecting the effect of the pH of the solution on the presence of RNA inside the VLP by agarose gel electrophoresis and EB staining.

The results showed that the RNA content inside the VLP was stable within the pH range of 4.5-8.5 and began to decrease at pH 9.5. The RNA inside the VLP decreased significantly at pH 10.5 and above, and the RNA could not be detected at pH 11.0. The appearance of RNA outside VLP indicated that RNA would be released from inside the VLP under such alkaline conditions (see FIG. 12). The RNA inside the VLP of PLA plays a key role in the B cell-related immune activation mechanism of PLA (Sheng Hong et al., B Cells Are the Dominant Antigen-Presenting Cells that Activate Naive CD4+ T Cells upon Immunization with a Virus-Derived Nanoparticle Antigen, Immunity, 2018.10, 49:1-14). The test found that when there is RNA inside the VLP of PLA, the RNA acts as a TLR stimulator, enabling PLA to work by relying on B cell-related immune mechanisms, and the immune effect is better than that of PLA without RNA inside the VLP. Therefore, the inventor proposed that the purification process of VLP needs to be under suitable pH conditions, such as pH 4.0-9.0, and strong alkaline conditions above pH 10.5 should be avoided.

### Example 6: Ability of PLA-SARS-CoV2 Vaccine to Induce Anti-Novel Coronavirus RBD Antibodies

C57BL/6 mice (purchased from SPF) were divided into four groups: (1) RBD antigen mixed with aluminum adjuvant (Alum, purchased from Pierce), 12 mice, 10 μg/mouse; (2) RBD antigen mixed with CpG1826 adjuvant (sequence is tccatgacgttcctgacgtt), 4 mice, 10 μg/mouse (the amount of CpG is 50 μg/mouse); (3) extracellular segment of S protein mixed with aluminum adjuvant, 4 mice, 50 μg/mouse; (4) PLA-SARS-CoV2 (i.e., the vaccine complex formed by connecting the VLP formed after AP205 modification and the SARS CoV2 RBD antigen, the same below), 21 mice, 10 μg/mice. Intraperitoneal immunization was used. Blood was collected 14 days after the first immunization, which was recorded as the first immunization serum. The second immunization was carried out 21 days after the first immunization, and blood was collected 7 days after the second immunization (i.e., 28 days after the first immunization), which was recorded as the second immunization serum.

RBD-specific antibody response was detected by ELISA. The coating amount of RBD antigen was 2 μg/ml, 50 μl/well, and kept overnight at 4° C. The serum was diluted in a gradient (the initial dilution of the serum was 1:1000, and then continued to be diluted 5 times, and a total of 8 gradients were made), followed by incubating with the RBD-coated Elisa 96-well plate at room temperature for 3 hours. Secondary antibody IgG-HRP (Bethyl Laboratories) was incubated at room temperature for 1 hour. After color development, the OD value of the corresponding well was read in a microplate reader. The wells without serum incubation were taken as the blank control. The average value of the OD values of 4-8 blank control wells plus 10 times the standard deviation is the reference value, and the lowest dilution of serum greater than the reference value is recorded as the antibody titer.

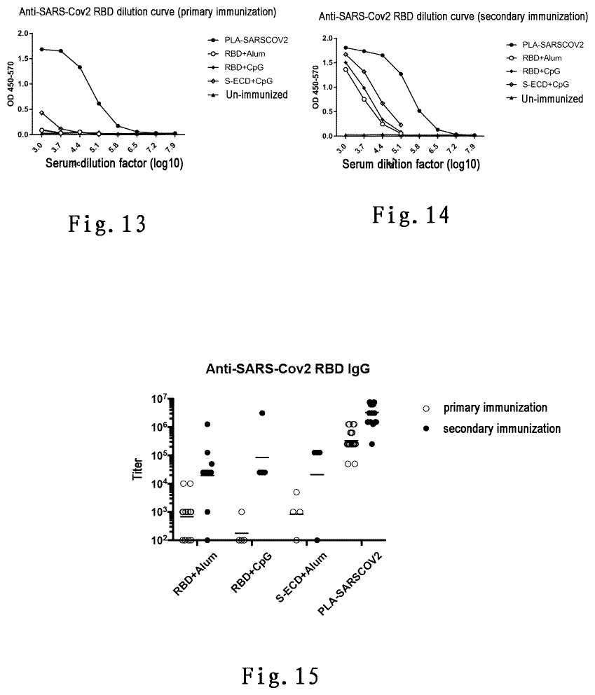

Regarding the specific measured values of different groups, the vertical axis is the OD reading value, and the abscissa axis is the Log value of serum dilution. It could be seen that the mice can produce a higher titer of RBD IgG antibody after one immunization (see FIG. 13). After the second immunization, the RBD IgG antibody titer can reach about 3×10⁶ (see FIG. 14). Compared with that after immunization with RBD antigen mixed with aluminum adjuvant, RBD antigen mixed with CpG adjuvant, and S protein extracellular segment of novel coronavirus mixed with aluminum adjuvant, the titer of the RBD IgG type antibody produced by PLA-SARS-CoV2 vaccine after the initial immunization or re-immunization could be increased by about 100 times (see FIG. 15).

### Example 7: Neutralizing Antibodies Produced in Mice Immunized with PLA-SARS-CoV2 Vaccine

Using RBD antigen mixed with aluminum adjuvant, RBD antigen mixed with CpG1826 adjuvant, extracellular segment of S protein mixed with aluminum adjuvant, and PLA-SARS-CoV2, induction of neutralizing antibody was compared by the following neutralizing antibody detection method as follows: serum dilution in 300 μl 2% DMEM medium, 3-fold dilution. 200 μl of serum at different dilutions were incubated with MOI 0.01 live virus (10 μl) at 37° C. for 1 hour. 200 μl was used to infect VERO-E6 cells in a 48-well plate. The medium was changed after 1 hour, and the cells were cultured in 2% DMEM medium for 24 hours. 150 μl supernatant was collected to extract RNA by MiniBEST Viral RNA/DNA Extraction Kit (Takara), and cDNA was reverse transcribed by PrimeScript™ RT reagent Kit with gDNA Eraser (Takara). The copy number was measured by the standard curve method (ABI 7500 (Takara TB Green® Premix Ex Taq II)) with the primers targeting the S gene.

Upstream primer (5'-3'):
(SEQ ID NO: 19)
CAATGGTTTAACAGGCACAGG;
Downstream primer (5'-3'):
(SEQ ID NO: 20)
CTCAAGTGTCTGTGGATCACG.

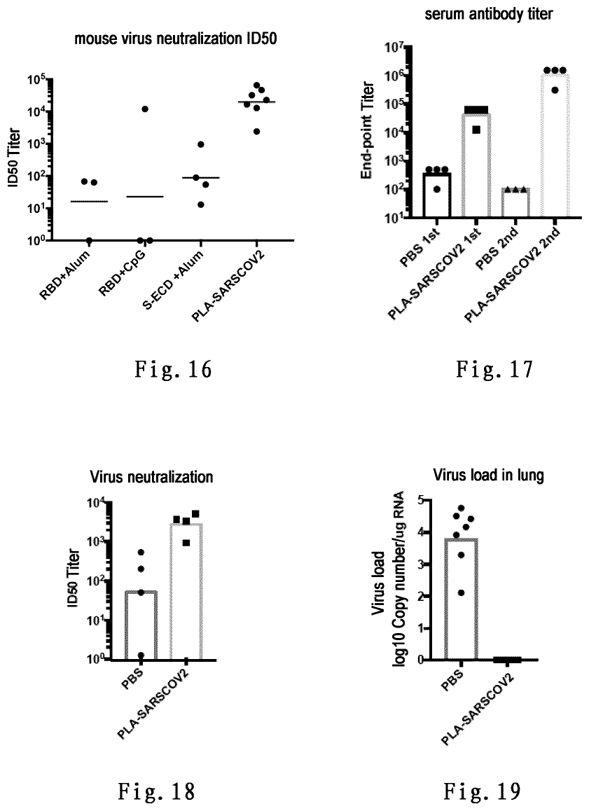

The vertical axis is the neutralizing antibody titer (ID50 titer) detected by ELISA, which shows that the level of neutralizing antibody induced by PLA-SARS-CoV2 of the present invention is 100 times higher than that of other traditional vaccines mixed with adjuvants (see FIG. 16).

### Example 8: Anti-RBD IgG Antibodies Produced in Macaques Immunized with PLA-SARS-CoV2 Vaccine

Eight young healthy macaques (male, 3-6 years old, all from Kunming Primate Research Center, Chinese Academy of Sciences) were used in the experiment. The immunization test was divided into two groups (4 for each) and each received intramuscular injection of PLA-SARS-CoV2 (20 μg/macaque/time) or normal saline (PBS, control group) twice (interval of 3 weeks). Blood was collected 14 days after the first injection and 7 days after the second injection, followed by serum separation. For the method of detecting anti-RBD IgG antibody titer, refer to the ELISA method described in Example 6. The secondary antibody was replaced with HRR-labeled goat anti-monkey IgG (purchased from Abcam, Cat. No. ab112767). FIG. 17 shows the anti-RBD IgG antibody levels in the serum 14 days after the initial immunization (1st) and 7 days after the second immunization (2nd) (the vertical axis is the antibody titer detected by ELISA), indicating that the anti-RBD IgG antibody level in the serum at the time of initial immunization was more than 100 times that of the PBS control, and the anti-RBD IgG antibody level after re-immunization was more than 1000 times higher than that of the PBS control.

### Example 9: Neutralizing Antibodies Produced in Macaques Immunized with PLA-SARS-CoV2 Vaccine

The inventors further tested the neutralizing antibodies induced by PLA-SARS-CoV2 vaccine of the present invention in macaques. The immunization process and conditions were the same as those in Example 8 above. The method of detecting serum neutralizing antibody titer was the same as that in Example 7. FIG. 18 shows that the level of neutralizing antibodies to the novel coronavirus in the serum after 7 days of re-immunization (2nd) increased dozens of times relative to the PBS control.

### Example 10: Virus Load in the Lung of Macaques Immunized with PLA-SARS-CoV2 Vaccine

The inventors next tested the virus load in the lung of macaques immunized with the PLA-SARS-CoV2 vaccine. The process of immunizing macaques with PLA-SARS-CoV2 vaccine was the same as that in Example 8. The virus used in the experiment was Novel Coronavirus 107 strains (provided by the Guangdong Provincial Center for Disease Control and Prevention in China). The virus strains were expanded by Vero-E6 cell lines, and half of the tissue culture infection dose was determined by the Reed-Muench method.

Viral challenge experiments were performed 10 days after re-immunization. The way of challenge is a combination method, intranasal (0.4 mL/nostril) and intratracheal (1.2 mL, fiberoptic bronchoscope). The total virus titer was 1×10⁷ TCID50 mL, which was diluted with sterile 0.9% normal saline.

The virus load in lung of the two groups of animals after 7 days was detected by RT-PCR. Total RNA was extracted from swabs and tracheal brushes using a kit (Roche Germany), and RNA from tissue samples was extracted using TRIzol reagent (Thermo USA). Viral RNA was detected using a probe-one-step real-time quantitative PCR kit (TOYOBO, Japan). The primers and probe are as follows: upstream primer 5′-GGGGAACTTCTCCTGCTAGAAT-3′ (SEQ ID NO: 21), downstream primer 5′-CAGACATTTTGCTCTCCAAGCTG-3′ (SEQ ID NO: 22) and FAM-TTGCTGCTGCTTGACAGATT-TAMRA-3′ (SEQ ID NO: 23). Each test sample was diluted according to the standards of the National Institute of Metrology of China, and finally the copy number of each sample was calculated.

The results showed that the virus in the lung of the macaque almost completely disappeared after immunization with the PLA-SARS-CoV2 vaccine of the present invention (FIG. 19, the vertical axis of the figure shows the logarithmic value of the virus copy number per μg RNA). Moreover, according to the results of induced specific antibodies and neutralizing antibodies by the PLA-SARS-CoV2 vaccine of the present invention in the foregoing examples, it can be reasonably inferred that the vaccine of the present invention would have significantly better results in the challenge test compared to other traditional vaccines that require additional adjuvants.

### Example 11: Th1-Biased Immune Response Induced by PLA-SARS-CoV2 Vaccine

The inventors tested the Th1-biased immune response induced by PLA-SARS-CoV2 vaccine of the present invention in mice. C57BL/6 mice (purchased from Speyford) were divided into three groups: (1) RBD antigen mixed with aluminum adjuvant (Alum, purchased from Pierce), 6 mice, 10 μg/mouse; (2) RBD antigen mixed with CpG1826 adjuvant (sequence is tccatgacgttcctgacgtt) and aluminum adjuvant (Alum, purchased from Pierce), 8 mice, 10 μg/mouse (the amount of CpG is 50 μg/mouse); (3) PLA-SARS-CoV2, 10-18 mice, 10 μg/mouse. Intraperitoneal immunization was used. The second immunization was carried out 21 days after the first immunization, and the blood was collected 7 days after the second immunization (i.e., 28 days after the first immunization), and recorded as the second immunization serum. The second immunization serum was used for the following experiments.

The isotype of the RBD-specific antibody was detected using ELISA. The coating amount of RBD antigen was 2 μg/ml, 50 μl/well, and kept overnight at 4° C. The serum was diluted in a gradient (the initial dilution of the serum was 1:1000, and then continued to be diluted 5 times, and a total of 8 gradients were made), followed by incubating with the RBD-coated Elisa 96-well plate at room temperature for 3 hours. The above-mentioned plates were divided into 6 groups, to detect the isotypes of different RBD-specific antibodies. Each group was incubated with the secondary antibody IgG-HRP (Bethyl Laboratories), the secondary antibody IgA-HRP (Bethyl Laboratories), and the secondary antibody IgM-HRP (Southern Biotech), secondary antibody IgG1-HRP (Southern Biotech), secondary antibody IgG2b-HRP (Southern Biotech), secondary antibody IgG2a/c-HRP (Southern Biotech), secondary antibody IgG3-HRP (Southern Biotech) at room temperature for 1 hour. After color development, the OD value of the corresponding well was read in a microplate reader. The wells without serum incubation were taken as the blank control. The average value of the OD values of 4-8 blank control wells plus 10 times the standard deviation is the reference value, and the lowest dilution of serum greater than the reference value is recorded as the antibody titer.

On the other hand, the inventors also used ELISpot (enzyme-linked immunosorbent spot) to detect IFNγ-secreting cells to test whether the PLA-SARS-CoV2 vaccine of the present invention induced a Th1-biased immune response in mice.

C57BL/6 mice (purchased from Speyford) were divided into three groups: (1) RBD antigen mixed with aluminum adjuvant (Alum, purchased from Pierce), mice, 10 μg/mouse; (2) PLA-SARS-CoV2, 10 mice, 10 μg/mouse. Intraperitoneal immunization was used. The second immunization was carried out 21 days after the first immunization, and the mice were dissected 7 days after the second immunization (i.e., 28 days after the first immunization), and spleen cells were obtained; (3) unimmunized mice were used as negative controls, 4 mice.

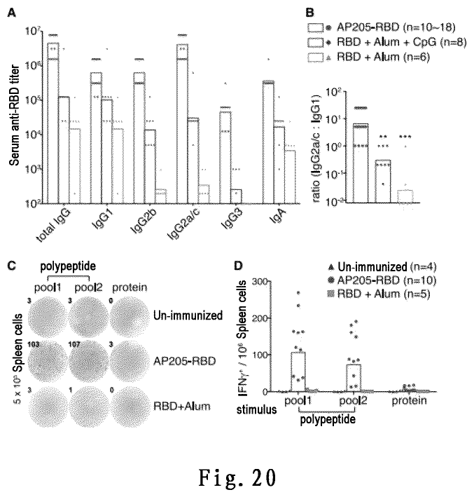

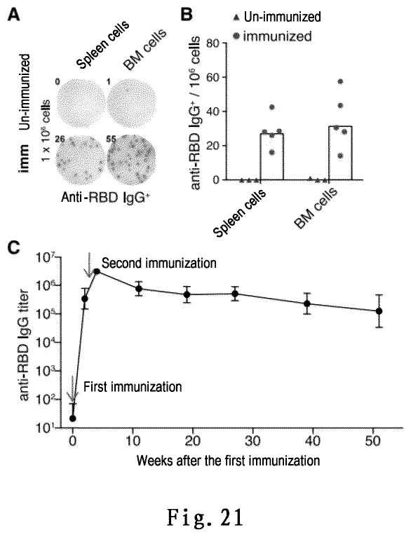

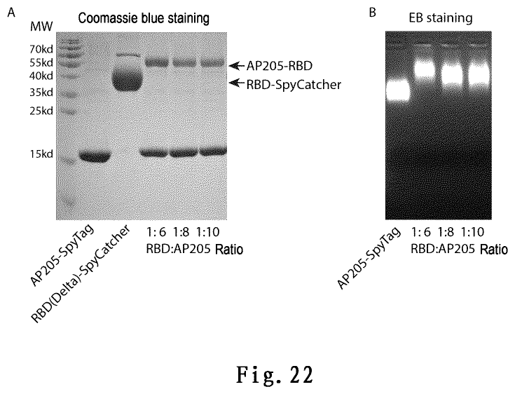

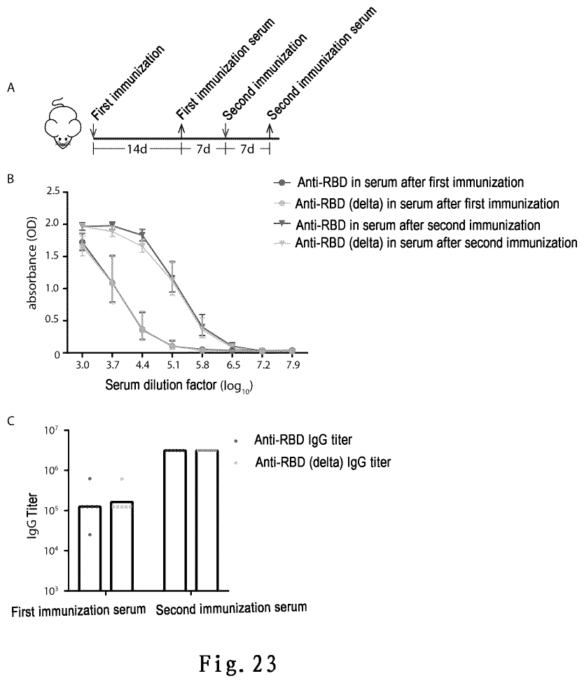

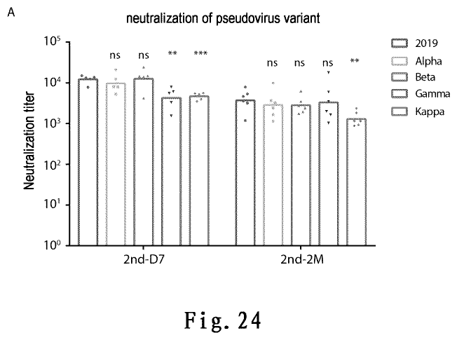

Different RBD fragments of 15-mer were incubated in plates precoated with IFNγ antibody (clone AN-18, Invitrogen) for 20 hours at 37° C. As shown in FIG. 20C-D, in pool 1, amino acid fragments of 420-434, 426-440 and 445-459 aa of the S protein from SARS-CoV-2 were used for stimulation incubation; in pool 2, amino acid fragments of 511-525, 517-531 and 535-549 aa of the S protein from SARS-CoV-2 were used for stimulation incubation; and in protein, the intact RBD protein from SARS-CoV-2 was used for stimulation incubation. Then Biotin-labeled IFNγ antibody (clone R4-6A2, Invitrogen) was used for incubation at room temperature for 3 hours, followed by using HRP-strepavidin (Jackson ImmunoResearch, USA) to incubate at room temperature for 1 hour. Spots were detected using an ImmunoSpot analyzer (Cellular Technology Limited, USA), and the number of spots was counted.

Results:

Intracellular pathogens such as viruses usually require a Th1-biased immune response to effectively clear the pathogen. Interferon gamma (IFNγ), a characteristic cytokine produced by Th1, is also produced by cytotoxic CD8+ T cells. From a B cell perspective, cytokines associated with Th1 responses tended to promote Ig isotype switching to IgG2a/c in mice, comparable to IgG1 in humans, whereas Th2-related cytokines promoted Ig isotype switching to IgG1, comparable to IgG4 in humans. Th1-associated IgG subclasses are more effective at mediating antibody-dependent cellular cytotoxicity and phagocytosis than Th2-associated IgG subclasses, thus promoting antiviral responses more effectively.

To determine whether AP205-RBD-induced responses were Th1-biased, the inventors first examined the Ig isotype of anti-RBD antibodies. As expected, AP205-RBD induced high titers of IgG2a/c (FIG. 20A) and the ratio of IgG2a/c to IgG1 were significantly higher in the AP205-RBD immunized group than those in the soluble RBD immunized group (FIG. 20B). Addition of CpG on top of aluminum adjuvant did increase the ratio of the IgG2a/c to IgG1 in the soluble RBD immunized group as expected, but not to the same level as that of the AP205-RBD immunized group (FIG. 20B).

To further assess whether AP205-RBD induces a Th1 response, the inventors examined IFNγ-secreting cells in splenocytes from naive or immunized mice. 15-mer peptides from RBD sequences or intact RBD proteins were used for in vitro stimulation. In mice immunized with AP205-RBD, many cells secreted IFNγ after peptide stimulation, whereas few of these cells were found in naive or mice immunized with soluble RBD plus aluminum adjuvant (FIGS. 20C and 20D), suggesting that AP205-RBD did induce a Th1-biased response.

Overall, IgG2a/c dominated the antibody response, and the production of many IFNγ-secreting cells supported that AP205-RBD induced a Th1-biased response.

### Example 12: Persistent Humoral Memory Induced by PLA-SARS-CoV2 Vaccine

C57BL/6 mice (purchased from SPF) were immunized with PLA-SARS-CoV2 vaccine. Intraperitoneal immunization was used. Mice were immunized twice using the method of Example 6. The RBD IgG antibody titer of the mouse serum was detected according to the method of Example 6 at different time points.

To assess AP205-RBD-induced humoral memory, the inventors examined RBD-specific long-lived plasma cells (PCs) from mice immunized 3-4 months earlier. Because long-lived PCs are found in bone marrow cells (BM) and can also be found in the spleen, RBD-specific IgG-secreting cells of splenocytes and BM cells were detected by ELISpot. Indeed, RBD-specific PCs were abundant in spleen and BM even 4 months after immunization (FIGS. 21A and 21B). The inventors consistently found that anti-RBD IgG was relatively stable from 2 months up to a year after the second immunization (FIG. 21C).

It could be seen that the PLA-SARS-CoV2 vaccine could maintain long-lasting immune memory for at least about 1 year.

### Example 13: Construction of Fusion Protein of SpyCatcher-RBD (Delta) Mutant

The delta mutant has two mutation sites in RBD, namely L452R and T478K. The above two sites were mutated by point mutation PCR on the basis of the original strain SpyCatcher-RBD. Two pairs of primers were designed as follows:

SCRBDL452R-F:
(SEQ ID NO: 25)
GGTAACTATAACTATAGATATAGACTGT
SCRBDL452R-R:
(SEQ ID NO: 26)
TCTATAGTTATAGTTACCCTCCCACCTTA
SCRBDT478K-F:
(SEQ ID NO: 27)
CTATCAGGCTGGATTCaagCCTTGTAACG
SCRBDT478K-R:
(SEQ ID NO: 28)
cttAGATCCAGCCTGATAGATCTCGGTAG

Using the original strain SpyCatcher-RBD as a template, high-fidelity DNA polymerase Phusion (Thermo scientific F-530S) was used for point mutation PCR. Then 2 μL DpnI endonuclease (Tarara 1235S) was added to 50 μL PCR system and kept in water bath at 37° C. for 2 hours to digest the template plasmid with methylation modification, and then the product obtained by PCR was purified using a small amount of DNA product purification kit (Zhuangmeng Bio ZP201-3). 10 μl of the ligation reaction solution of the purified product was added to 150 μl of XLI-Blue competent Escherichia coli (Transgene CD401-02), and kept on ice for 30 minutes, followed by heat shock at 42° C. for 1 minute. 100 μl was pipetted on the ampicillin-resistant LB plate. The plate was incubated at 37° C. for 14-16 hours. The plasmid DNA was extracted with a plasmid mini-extraction kit (Tiangen DP103-03) and verified by sequencing, and the expression plasmid of the mutated SpyCatcher-RBD (delta) was successfully constructed.

### Example 14: Purification and Preparation of SpyCatcher-RBD (Delta) Mutant Fusion Protein

The experimental method was the same as the expression method in Example 2, i.e., the same as the method for expressing the wild-type original strain SpyCatcher-RBD fusion protein.

### Example 15: Construction of PLA-SARS-CoV2 (Delta) Candidate Vaccine

The SpyCatcher-RBD (delta) was connected to the virus-like particle AP205 through the covalent connection between SpyCatcher and SpyTag using the same method as that of the wild-type original strain PLA.

The process and conditions of the connection reaction are the same as those of the wild-type original strain, i.e., the conditions in Example 2(3).

The specific measurement process and conditions are:
(1) The specific process and conditions of the protein gel: the loading amount was 10 μg PLA or the connected product, 5% upper glue, 15% lower glue, 80 volts for 30 minutes, 130 volts for 50 minutes. The results are shown in FIG. 22A.
(2) The specific process and conditions of the nucleic acid gel: the loading amount was 10 μg PLA or the connected product, 1% nucleic acid gel, 90 volts for 20 minutes. The results are shown in FIG. 22B.

By detecting the connection reaction by protein gel and detecting the nucleic acid inside the virus-like particles by nucleic acid gel, it was found that the connection ratios for the SpyCatcher-RBD (delta) mutant or the original strain was close to the range of 1:6 to 1:10, the connection reaction was complete and the nucleic acids inside the particle remained intact after connection.

### Example 16: Immunogenicity Detection of PLA-SARS-CoV2 (Delta) Candidate Vaccine

Experimental animals: C57BL/6 mice (purchased from SPF), half male and half male
Immunization dose: 10 μg/mouse
Immunization method: the PLA-SARS-CoV2 (delta) candidate vaccine constructed above was used for intraperitoneal immunization. Blood was collected 14 days after the first immunization, which was recorded as the first immunization serum. The second immunization was carried out 21 days after the first immunization, and blood was collected 7 days after the second immunization (i.e., 28 days after the first immunization), which was recorded as the second immunization serum, as shown in FIG. 23A.

The binding ability of mouse serum after immunication with PLA-SARS-CoV2 (delta) candidate vaccine against RBD delta mutant and original RBD protein was tested by enzyme-linked immunosorbent assay (ELISA).

The experimental method is as follows: the antigen was diluted with carbonate antigen coating solution to 2 μg/mL, 50 μL/well was added to the ELISA plate (Costar 3690) and was incubated overnight at 4° C. Then the coating solution was discarded, and it was washed 3 times with washing solution (PBS+0.05% Tween20) by the ELISA washer (Nunc Immuno-washer). 100 μL of blocking solution (PBS+1% BSA+0.05% Tween20) was added to each well, and it was blocked for 1 hour at room temperature. The serum to be tested was diluted during this period. The initial dilution of the serum was 1:1000, and then it was continued to be diluted 5 times, and a total of 8 gradients were made. The blocking solution was discarded, and it was washed 3 times with the washing solution. 50 μL of the diluted primary antiserum was added to each well, and it was incubated at room temperature for 3 hours. The primary antibody was discarded, and it was washed 5 times. 50 μL diluted HRP-labeled IgG secondary antibody was added to each well, and it was incubated at room temperature for 1 hour. The secondary antibody was discarded, and it was washed 7 times. 50 μL of chromogenic solution TMB (0.1 M sodium acetate solution at pH 5.5+1% of 10 mg/mL TMB (Sigma-Aldrich, 860336-1G) and 0.1% of 30% H2O2) was added to each well, and 25 μL 2N H₂SO₄ was added to each well to terminate the reaction until the color no longer deepened. The absorbance at 450 nm and 570 nm was read on a microplate reader. The wells without serum incubation were taken as the blank control. The average value of the OD values of 4-8 blank control wells plus 10 times the standard deviation is the reference value, and the lowest dilution of serum greater than the reference value is recorded as the antibody titer. ELISA results are shown in FIGS. 23B-C. The vertical axis in FIG. 23B is the OD reading, and the abscissa axis is the log value of the serum dilution.

The results showed, it could be seen that after immunization with the PLA-SARS-CoV2 (delta) candidate vaccine constructed above, its response to RBD (delta) and the original RBD is basically the same, indicating that the PLA-SARS-CoV2 (delta) candidate vaccine can induce a good RBD (delta) specific IgG antibody, and also has good reactivity to original RBD. The vertical axis of FIG. 23C is the antibody titer, and each point in the figure represents the serum antibody titer level of a mouse. The PLA-SARS-CoV2 (delta) candidate vaccine can induce good RBD (delta)-specific IgG antibody with a titer of about 2×10\hat{ }5, after one immunization. After the second immunization, the titer of RBD (delta)-specific IgG antibody further increased to about 3×10\hat{ }6. FIG. 23C columns, from left to right, are anti-RBD IgG titer and anti-RBD (delta) IgG titer.

### Example 17: The Neutralizing Response of Serum Immunized with PLA-SARS-CoV2 Vaccine Against Different Mutant Pseudoviruses

The inventors also tested the neutralizing response of the original strain of PLA-SARS-CoV2 vaccine (i.e., the PLA-SARS-CoV2 vaccine used in Example 6) against different mutant pseudoviruses. The immunization method was the same as above, and the mouse serum was collected 7 days after the second immunization and two months after the second immunization, and the neutralization of different mutant pseudoviruses was detected.

The experimental method is as follows: the HIV backbone virus reporter vector luciferase expression vector (pNL43R-E-luciferase, Invitrogene) and pcDNA3.1 (Invitrogen) encoding the spike protein of different mutants were co-transfected into 293T cells to obtain pseudoviruses. The supernatant containing the pseudovirus was collected 48 hours after transfection. Pseudovirus titers were determined using luciferase activity in relative light units (Bright-Glo Luciferase Assay Vector System, Promega Biosciences, USA). In the neutralization experiment, different mutant pseudoviruses were incubated with serially diluted mouse serum at 37° C. for one hour. Then Huh7 cells were added and three replicates (approximately 1.5×10\hat{ }4/well) were set up. Luciferase activity was detected after 48 hours. GraphPad Prism curve fitting was used to determine the half maximal inhibitory concentration (IC50) of the serum, and this was used to represent the neutralization titer of the serum against different mutant pseudoviruses. The results of pseudovirus neutralization are shown in FIG. 24. The columns from left to right are 2019, Alpha, Beta, Gamma, Kappa.

The results showed that, the serum of mice immunized with PLA-SARS-CoV2 vaccine had good neutralizing activity against the original strain of the novel coronavirus, with a neutralizing titer of about 1×10\hat{ }4. The duration was prolonged, and it was still maintained at a high level two months after the second immunization. At the same time, it was found that the serum of mice immunized with PLA-SARS-CoV2 vaccine also had good neutralizing activity against the mutant pseudovirus, the neutralizing activity against the Alpha strain and the Beta strain was basically the same as that of the original strain, and the neutralization activity against the Gamma strain and the Kappa strain decreased. As time went on, the difference between the neutralizing activity against the two mutant strains and the neutralizing activity against the original strain was reducing, indicating that as the immune response progressed, the PLA-SARS-CoV2 vaccine could induce broader spectrum of neutralizing antibodies.

1. A soluble pathogen-like antigen complex comprising:
(1) a virus-like particle (VLP), which is self-assembled by a first fusion protein comprising a viral capsid protein or a variant thereof at its N-terminus and SpyTag at its C-terminus,
(2) a second fusion protein, comprising an antigen from S1 protein of SARS-CoV2 virus or a variant thereof and SpyCatcher, preferably the SpyCatcher is at the N-terminus of the second fusion protein;
wherein, the virus-like particle also encapsulates nucleic acid inside it, and
wherein the virus-like particle and the antigen from the S1 protein of SARS-CoV2 virus are covalently connected through the SpyCatcher and the SpyTag, so that the antigen from S1 protein of SARS-CoV2 virus or a variant thereof is displayed on the surface of the virus-like particle.

2. The soluble pathogen-like antigen complex according to claim 1, wherein the nucleic acid encapsulated in the virus-like particle is the nucleic acid which is encapsulated by the virus-like particle when self-assembling and is from host bacteria used to express the virus-like particles, preferably the host bacteria is Escherichia coli, and preferably the nucleic acid is RNA.

3. The soluble pathogen-like antigen complex according to claim 1, wherein the capsid protein is from Escherichia coli phage Qβ, MS2 or AP205, preferably from Escherichia coli phage AP205.

4. The soluble pathogen-like antigen complex according to claim 3, wherein the sequence of the capsid protein of the phage AP205 has at least 90%, 91%, 92%, 93%, 94%, 95%, 96%, 97%, 98%, 99% or 100% identity with SEQ ID NO: 1.

5. The soluble pathogen-like antigen complex according to claim 4, wherein the sequence of the capsid protein of the phage AP205 is SEQ ID NO: 1.

6. The soluble pathogen-like antigen complex according to claim 1, wherein the antigen is a RBD sequence of the S1 protein of SARS-CoV2 virus.

7. The soluble pathogen-like antigen complex according to claim 6, wherein the sequence of the antigen has at least 90%, 91%, 92%, 93%, 94%, 95%, 96%, 97% %, 98%, 99% or 100% identity with SEQ ID NO: 2.

8. The soluble pathogen-like antigen complex according to claim 7, wherein the sequence of the antigen is SEQ ID NO: 2 or SEQ ID NO: 24.

9. The soluble pathogen-like antigen complex according to claim 1, wherein the sequence of the SpyTag has at least 90%, 91%, 92%, 93%, 94%, 95%, 96%, 97%, 98%, 99% or 100% identity with of SEQ ID NO: 3, and the sequence of the SpyCatcher has at least 90%, 91%, 92%, 93%, 94%, 95%, 96%, 97%, 98%, 99% or 100% identity with SEQ ID NO: 4.

10. The soluble pathogen-like antigen complex according to claim 1, wherein in the first fusion protein, the phage capsid protein or a variant thereof is linked with the SpyTag via a first linker peptide, and in the second fusion protein, the antigen or a variant thereof is linked to the SpyCatcher via a second linker peptide.

11. The soluble pathogen-like antigen complex according to claim 10, wherein the sequence of the first linker peptide is SEQ ID NO: 5, and the sequence of the second linker peptide is SEQ ID NO: 6.

12. The soluble pathogen-like antigen complex according to claim 1, wherein the second fusion protein is connected to the virus-like particle at a ratio of less than or equal to 1:1, preferably connected at a ratio of 1:6˜1:12, and preferably connected at a ratio of 1:6, 1:7, 1:8, 1:9, 1:10, so as to ensure the solubility and immunogenicity of the pathogen-like antigen complex, the ratio is calculated as the ratio of the SpyCatcher on the second fusion protein to the SpyTag on the virus-like particle.

13. A pathogen-like antigen vaccine composition, comprising the soluble pathogen-like antigen complex according to claim 1 and pharmaceutically acceptable carriers and/or excipients.

14. A method for preventing and/or treating diseases associated with SARS-CoV2 virus infection in a subject in need thereof, wherein a preventive and/or therapeutically effective amount of the soluble pathogen-like antigen complex or vaccine composition of claim 1 is administered to the subject.


SEQUENCE LISTING (28 sequences)

SEQ ID NO: 1 - protein, 136 aa, Artificial Sequence
  AP205 amino acid sequence
MEFGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 2 - protein, 223 aa, Artificial Sequence
  Covid-19 RBD amino acid sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 3 - protein, 15 aa, Artificial Sequence
  ST amino acid sequence
AHIVMVDAYKPTKTG

SEQ ID NO: 4 - protein, 92 aa, Artificial Sequence
  SC amino acid sequence
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 5 - protein, 11 aa, Artificial Sequence
  first linker peptide amino acid sequence
GSGGSGTGGSG

SEQ ID NO: 6 - protein, 6 aa, Artificial Sequence
  second linker peptide amino acid sequence
GSGGSG

SEQ ID NO: 7 - protein, 162 aa, Artificial Sequence
  AP205-first linker peptide-ST amino acid sequence
MEFGSMANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTAGSGGSGTGGSGAHIVMVDAYKPTKTG

SEQ ID NO: 8 - protein, 321 aa, Artificial Sequence
  SC-second linker peptide-RBD amino acid sequence
DSATHIKFSKRDEDGKELAGATMELRDSSGKTISTWISDGQVKDFYLYPGKYTFVETAAPDGYEVATAITFTVNEQGQVTVNGKATKGDAHIGSGGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNF

SEQ ID NO: 9 - DNA, 393 bp, Artificial Sequence
  AP205 cDNA sequence
atggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgct

SEQ ID NO: 10 - DNA, 276 bp, Artificial Sequence
  SC cDNA sequence
gatagcgctacccatatcaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatatt

SEQ ID NO: 11 - DNA, 1068 bp, Artificial Sequence
  RBD-SC cDNA sequence
atgttcgtgtttctggtgctgctgcctctggtgagctcccagtgcaccggtagggtgcagcctaccgagtctatcgtgcgcttcccaaacatcacaaatctgtgccccttcggcgaggtgtttaatgccacccggtttgccagcgtgtatgcctggaatcggaagagaatctccaactgcgtggccgactattctgtgctgtacaacagcgccagcttcagcaccttcaagtgctatggcgtgtcccccaccaagctgaatgacctgtgcttcacaaacgtgtacgccgactcttttgtgatccggggcgatgaggtgagacagatcgcaccaggacagaccggcaagatcgcagactacaattataagctgcctgacgatttcacaggctgcgtgatcgcctggaacagcaacaatctggattccaaagtgggcggcaactacaattatctgtacaggctgttccgcaagtccaatctgaagccatttgagagggacatcagcaccgagatctaccaggccggctccacaccctgcaatggcgtggagggcttcaactgttattttcctctgcagagctacggcttccagccaaccaacggcgtgggctatcagccctaccgcgtggtggtgctgtcctttgagctgctgcacgcaccagcaaccgtgtgcggacctaagaagtctacaaacctggtgaagaataagagcgtgaactttggaggaggaggaggaggaagcaagcttgactctgccacccacatcaagttcagcaagagggacgaggatggcaaggagctggcaggagcaacaatggagctgagagatagctccggcaagaccatctccacatggatctctgacggccaggtgaaggatttctacctgtatcctggcaagtatacctttgtggagacagcagcaccagacggatacgaggtggcaaccgccatcacctttacagtgaatgagcagggccaggtgaccgtgaacggcaaggccacaaagggcgatgcacacatcggaggaagccatcatcatcatcatcaccatcatcatcatcatcac

SEQ ID NO: 12 - DNA, 1059 bp, Artificial Sequence
  SC-RBD cDNA sequence
atgttcgtgtttctggtgctgctgcctctggtgagctcccagtgcgactctgccacccacatcaagttcagcaagagggacgaggatggcaaggagctggcaggagcaacaatggagctgagagatagctccggcaagaccatctccacatggatctctgacggccaggtgaaggatttctacctgtatcctggcaagtatacctttgtggagacagcagcaccagacggatacgaggtggcaaccgccatcacctttacagtgaatgagcagggccaggtgaccgtgaacggcaaggccacaaagggcgatgcacacatcggatctggaggaagcggaagggtgcagcctaccgagtctatcgtgcgcttcccaaacatcacaaatctgtgccccttcggcgaggtgtttaatgccacccggtttgccagcgtgtatgcctggaatcggaagagaatctccaactgcgtggccgactattctgtgctgtacaacagcgccagcttcagcaccttcaagtgctatggcgtgtcccccaccaagctgaatgacctgtgcttcacaaacgtgtacgccgactcttttgtgatccggggcgatgaggtgagacagatcgcaccaggacagaccggcaagatcgcagactacaattataagctgcctgacgatttcacaggctgcgtgatcgcctggaacagcaacaatctggattccaaagtgggcggcaactacaattatctgtacaggctgttccgcaagtccaatctgaagccatttgagagggacatcagcaccgagatctaccaggccggctccacaccctgcaatggcgtggagggcttcaactgttattttcctctgcagagctacggcttccagccaaccaacggcgtgggctatcagccctaccgcgtggtggtgctgtcctttgagctgctgcacgcaccagcaaccgtgtgcggacctaagaagtctacaaacctggtgaagaataagagcgtgaactttggaggaggaggaagccatcatcatcatcatcaccatcatcatcatcatcac

SEQ ID NO: 13 - DNA, 31 bp, Artificial Sequence
  SC-AP205 expression vector upstream primer sequence
acgggatccatgtcgtactaccatcaccatc

SEQ ID NO: 14 - DNA, 48 bp, Artificial Sequence
  SC-AP205 expression vector downstream primer sequence
cccggatccactgccgctacctccaatatgagcgtcacctttagttgc

SEQ ID NO: 15 - DNA, 31 bp, Artificial Sequence
  AP205-SC expression vector upstream primer sequence
acgaccggtatgtcgtactaccatcaccatc

SEQ ID NO: 16 - DNA, 33 bp, Artificial Sequence
  AP205-SC expression vector downstream primer sequence
cccaccggtaatatgagcgtcacctttagttgc

SEQ ID NO: 17 - DNA, 54 bp, Artificial Sequence
  AP205-ST expression vector upstream primer sequence
ccggtggtagcggcgcccacatcgtgatggtggacgcctacaagccgacgaaga

SEQ ID NO: 18 - DNA, 54 bp, Artificial Sequence
  AP205-ST expression vector downstream primer sequence
ccggtcttcgtcggcttgtaggcgtccaccatcacgatgtgggcgccgctacca

SEQ ID NO: 19 - DNA, 21 bp, Artificial Sequence
  the upstream primer sequence in Example 7
caatggtttaacaggcacagg

SEQ ID NO: 20 - DNA, 21 bp, Artificial Sequence
  the downstream primer sequence in Example 7
ctcaagtgtctgtggatcacg

SEQ ID NO: 21 - DNA, 22 bp, Artificial Sequence
  the upstream primer sequence in Example 10
ggggaacttctcctgctagaat

SEQ ID NO: 22 - DNA, 22 bp, Artificial Sequence
  the downstream primer sequence in Example 10
cagacattttgctctcaagctg

SEQ ID NO: 23 - DNA, 20 bp, Artificial Sequence
  the probe sequence in Example 10
ttgctgctgcttgacagatt

SEQ ID NO: 24 - protein, 321 aa, Artificial Sequence
  Covid-19 (delta) RBD amino acid sequence
DSATHIKFSKRDEDGKELAGATMELRDSSGKTISTWISDGQVKDFYLYPGKYTFVETAAPDGYEVATAITFTVNEQGQVTVNGKATKGDAHIGSGGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKSVNF

SEQ ID NO: 25 - DNA, 28 bp, Artificial Sequence
  SCRBDL452R-F in Example 13
ggtaactataactatagatatagactgt

SEQ ID NO: 26 - DNA, 28 bp, Artificial Sequence
  SCRBDL452R-R in Example 13
tctatagttatagttacctcccacctta

SEQ ID NO: 27 - DNA, 29 bp, Artificial Sequence
  SCRBDT478K-F in Example 13
ctatcaggctggatctaagccttgtaacg

SEQ ID NO: 28 - DNA, 29 bp, Artificial Sequence
  SCRBDT478K-R in Example 13
cttagatccagcctgatagatctcggtag